(19) United States
(12) Patent Application Publication
(10) Pub. No.: US 2017/0101684 A1
(43) Pub. Date: Apr. 13, 2017

(54) METHOD FOR DIAGNOSING AND TREATING MULTIPLE MYELOMA

(71) Applicants: Erasmus University Medical Center Rotterdam, Rotterdam (NL); Skylinedx B.V., Rotterdam (NL)

(72) Inventors: Martinus Hendrikus Van Vliet, 's-Gravendeel (NL); Pieter Sonneveld, Rotterdam (NL)

(73) Assignees: Erasmus University Medical Center Rotterdam, Rotterdam (NL); Skylinedx B.V., Rotterdam (NL)

(21) Appl. No.: 15/128,082

(22) PCT Filed: Mar. 28, 2015
(86) PCT No.: PCT/EP2015/056822

§ 371 Date: Sep. 21, 2016

(30) Foreign Application Priority Data
Mar. 28, 2014 (EP) 14162459.3

(51) Int. Cl. C12Q 1/68; A61K 45/06; A61K 31/69

(52) U.S. Cl. CPC C12Q 1/6886; A61K 31/69; A61K 45/06; C12Q 2600/158; C12Q 2600/106; C12Q 2600/118

(57) ABSTRACT

The disclosure is in the field of medical treatments and relates to the treatment of cancer, in particular, multiple myeloma (MM). Even more in particular, it provides means and methods for the improved treatment of certain subgroups of MM patients, more in particular, subjects with a poor prognosis. In a particular embodiment, the disclosure provides a method of treatment wherein subjects with a poor prognosis are selected and treated with a proteasome inhibitor such as Bortezomib. The disclosure further provides means and methods for identifying subjects with a poor prognosis. More in particular, the disclosure provides a composition comprising a proteasome inhibitor for use in the treatment of a subject with multiple myeloma when the subject has been diagnosed with an amp1q chromosomal aberration.

### CROSS-REFERENCE TO RELATED APPLICATIONS

This application is a national phase entry under 35 U.S.C. §371 of International Patent Application PCT/EP2015/056822, filed Mar. 28, 2015, designating the United States of America and published in English as International Patent Publication WO 2015/144929 A1 on Oct. 1, 2015, which claims the benefit under Article 8 of the Patent Cooperation Treaty to European Patent Application Serial No. 14162459.3, filed Mar. 28, 2014.

### TECHNICAL FIELD

The application is in the field of in vitro diagnostic methods and medical treatments and relates to the diagnosis and treatment of cancer, in particular, Multiple Myeloma (MM). Even more in particular, it provides means and methods for the improved treatment of certain subgroups of MM patients, more in particular, MM subjects with a poor prognosis of the disease. In a particular embodiment, the disclosure provides a method of treatment wherein subjects with a poor prognosis are first selected and subsequently treated with a proteasome inhibitor such as Bortezomib. The disclosure further provides means and methods for identifying subjects with a poor prognosis.

### BACKGROUND

Multiple myeloma, also known as plasma cell myeloma or Kahler's disease, is a cancer of plasma cells, a type of white blood cell normally responsible for producing antibodies. In multiple myeloma, collections of abnormal plasma cells accumulate in the bone marrow, where they interfere with the production of normal blood cells. Most cases of myeloma also feature the production of a paraprotein, an abnormal antibody that can cause kidney problems. Bone lesions and hypercalcemia (high blood calcium levels) are also often encountered.

Multiple Myeloma is known to be a heterogeneous disease; the prognosis and response to therapy of patients with Multiple Myeloma varies widely between patients. Unfortunately, the risk-determining factors for the prognosis and response to treatment remain largely unknown.

Significant effort has been directed toward the identification of the molecular genetic events leading to this malignancy with the goals of improving early detection and providing new therapeutic targets. Unlike most hematological malignancies and more similar to solid tissue neoplasms, MM genomes are typified by numerous structural and numerical chromosomal aberrations,^{[1, 2]} including t(4; 14), t(11; 14), t(14; 16), t(14; 20), amp1q, del13q, and del17p.

Reflecting the increasing genomic instability that characterizes disease progression, metaphase chromosomal abnormalities can be detected in only one-third of newly diagnosed patients but are evident in the majority of patients with end-stage disease.^{[3]} Yet, applying DNA content or interphase fluorescence in situ hybridization (FISH) analyses, aneuploidy and translocations are detectable in virtually all subjects with MM.^{[4, 5]} Gene expression profiling studies have revealed clusters of patients with distinct expression patterns, including a signature that identifies high-risk patients (EMC92/SKY92).^{[6]}

However, technologies and methodologies for assessing these markers have not been standardized yet. Lack of standardization hampers marker interpretation for individual patients as well as across cohorts, and limits the emerging strategies that combine these markers toward patient stratification and personalized medicine.

Determination of the genetic aberrations in MM may be helpful in predicting disease outcome. In particular, the amp1q aberration is associated with a poor prognosis.^{[7]} Amp1q is synonymous with gain1q and “chromosome 1q amplification,” as described in the ISCN guidelines for cytogenetic aberrations.^{[8]} It is defined therein as an intra-chromosomal low-level amplification of DNA sequences of chromosome 1q. In MM, amp1q has been reported to range in size between cytoband 1q10 to 1q44,^{[9, 10]} and all these variants are defined as amp1q and are considered to have prognostic value in MM.^{[7, 11-16]}

Amp1q is typically detected in multiple myeloma cancer cells by interphase FISH (iFISH) on plasma cells such as CD138-purified plasma cells, which may be obtained from the bone marrow of the patient.^{[14]}

In an iFISH analysis, a cloned, fluorescent DNA sequence specific for chromosome 1q (FISH probe) is hybridized to the interphase myeloma cell chromosomes. The number of fluorescent spots per cell nucleus corresponding to the FISH probe are counted under the microscope. The detection of supernumerary (more than two) fluorescent spots per nucleus belonging to chromosome 1q are interpreted as amp1q. Amp1q in multiple myeloma can also be detected by multiplex ligation-dependent probe amplification,^{[17]} by G-banding or R-banding techniques, by comparative genomic hybridization (CGH) such as array-CGH or equivalent DNA copy number aberration (CNA) techniques.^{[11]}

The resulting karyotype (normal or amp1q) is regarded as a prognostic factor in myeloma as cases with amp1q have been described to have a less favorable or even poor outcome,^{[7, 11-16]} but a causative relation between this aberration and specific treatment(s) remains unknown. The IMWG guideline^{[15]} proposes to use “lack of 1q21 gain” in identifying patients with a good prognosis.

However, no treatments have been described that impact the prognosis of multiple myeloma patients with chromosome 1q amplifications.

The present tests designed to detect amp1q suffer from a number of disadvantages. Current testing is laborious, can take weeks until clinical reporting, are subjective and depend on highly skilled personnel and expensive equipment (e.g., fluorescent microscopes). Moreover, on average, FISH results can only be produced in about 50% to 60% of all cases due to sample quality or quantity issues.

### BRIEF SUMMARY

The disclosure provides a novel way of diagnosing and treating multiple myeloma (MM) patients. The method of treatment comprises a first diagnostic step wherein a subject is selected according to a gene expression profile and a subsequent step wherein the subject is treated with a proteasome inhibitor.

The disclosure also provides a method for an improved detection of an amp1q aberration, which is particularly beneficial to classify an MM patient.

The diagnostic method provided herein is suitable for determining whether a subject with multiple myeloma has an amp1q chromosomal aberration, as well as determining whether a subject with multiple myeloma is likely to respond to a treatment with a proteasome inhibitor.

In one aspect, this disclosure provides a composition comprising a proteasome inhibitor for use in the treatment of a subject with multiple myeloma wherein the subject has been diagnosed as having an amp1q chromosomal aberration.

In another aspect, this disclosure provides a method for determining whether a subject has an amp1q chromosomal aberration, comprising determining in a nucleic acid sample of a subject the expression level of a number of N genes selected from the group consisting of the genes of Table 1, wherein N is at least 2, and establishing that the subject has an amp1q aberration in the case where at least two of the N genes are overexpressed.

In another aspect, the disclosure provides a method for determining whether a subject has an amp1q chromosomal aberration, comprising determining in a nucleic acid sample of a subject the expression level of a number of N genes selected from the group consisting of the genes of Table 1, wherein N is at least 3, and establishing that the subject has an amp1q aberration in the case where at least three of the N genes are overexpressed.

In another aspect, this disclosure provides a method for determining whether a subject has an amp1q chromosomal aberration, comprising determining in a nucleic acid sample of a subject the expression level of a number of N genes selected from the group consisting of the genes of Table 1, wherein N is at least 4, and establishing that the subject has an amp1q aberration in the case where at least four of the N genes are overexpressed.

In general, it can be stated that the disclosure provides a method for determining whether a subject has an amp1q chromosomal aberration, comprising determining in a nucleic acid sample of a subject the expression level of a number of N genes selected from the group consisting of the genes of Table 1, wherein N is at least Y, and establishing that the subject has an amp1q aberration in the case where at least Y of the N genes are overexpressed, wherein Y is an integer of 3 or above.

In another aspect, this disclosure provides a method for determining whether a subject has an amp1q chromosomal aberration, comprising determining in a nucleic acid sample of a subject the expression level of all genes of Table 1, and establishing that the subject has an amp1q aberration in the case where all genes are overexpressed.

In another aspect, the disclosure provides a method for determining whether a subject with multiple myeloma is likely to respond to a treatment with a proteasome inhibitor, comprising determining in a nucleic acid sample of a subject with multiple myeloma the expression level of a number of N genes selected from the group consisting of the genes of Table 1, wherein N is at least 2, and establishing that the subject is likely to respond to a treatment with a proteasome inhibitor in the case where at least two of the N genes are overexpressed.

In another aspect, this disclosure provides a method for determining whether a subject with multiple myeloma is likely to respond to a treatment with a proteasome inhibitor, comprising determining in a nucleic acid sample of a subject with multiple myeloma the expression level of a number of N genes selected from the group consisting of the genes of Table 1, wherein N is at least 3, and establishing that the subject is likely to respond to a treatment with a proteasome inhibitor in the case where at least three of the N genes are overexpressed.

In another aspect, the disclosure provides a method for determining whether a subject with multiple myeloma is likely to respond to a treatment with a proteasome inhibitor, comprising determining in a nucleic acid sample of a subject with multiple myeloma the expression level of a number of N genes selected from the group consisting of the genes of Table 1, wherein N is at least 4, and establishing that the subject is likely to respond to a treatment with a proteasome inhibitor in the case where at least four of the N genes are overexpressed.

In another aspect, this disclosure provides a method for determining whether a subject with multiple myeloma is likely to respond to a treatment with a proteasome inhibitor, comprising determining in a nucleic acid sample of a subject with multiple myeloma the expression level of all of the genes of Table 1, and establishing that the subject is likely to respond to a treatment with a proteasome inhibitor in the case where all genes are overexpressed.

In another aspect, this disclosure provides a composition comprising a proteasome inhibitor for use in the treatment of a subject with multiple myeloma as described above, wherein the subject has been diagnosed as having an amp1q chromosomal aberration by performing a method as described herein, or by performing a method for determining whether a subject with multiple myeloma is likely to respond to a treatment according to the disclosure as described above.

In another aspect, the disclosure provides a method for the treatment of a subject with multiple myeloma, comprising the steps of:
a) determining whether a subject with multiple myeloma is likely to respond to a treatment with a proteasome inhibitor by determining the presence in the subject of an amp1q chromosomal aberration, wherein the presence of the aberration indicates that the patient is likely to respond to the treatment;
b) administering to the subject a proteasome inhibitor in the case where the amp1q chromosomal aberration is present in the subject, and avoiding the administration of a proteasome inhibitor to the subject in the case where the amp1q chromosomal aberration is not present in the subject,
wherein, preferably, the step of determining the presence in the subject of an amp1q chromosomal aberration is performed by determining in a nucleic acid sample of the subject the normalized expression level of at least two genes, such as three or four genes selected from the group consisting of the genes of Table 1, and wherein an overexpression of at least two of the genes is indicative of the presence in the subject of an amp1q chromosomal aberration,
wherein, preferably, the proteasome inhibitor is selected from the group consisting of Bortezomib, Carfilzomib, MLN9708, Delanzomib, Oprozomib, AM-114, Marizomib TMC-95A, Curcusone-D and PI-1840, more preferably, wherein the proteasome inhibitor is Bortezomib, and
wherein the method optionally further comprises the administration to the subject wherein the amp1q chromosomal aberration is present of drugs selected from the group consisting of Melphalan, prednisone, doxorubicin, dexamethasone, immunomodulating drugs and monoclonal antibody drugs.

Preferred embodiments of these aspects will be described in more detail herein. For the purpose of clarity and a concise description, features are described herein as part of the same or separate embodiments; however, it will be appreciated that the scope of the disclosure may include embodiments having combinations of all or some of the features described.

### BRIEF DESCRIPTION OF THE DRAWINGS

FIG. 1: Kaplan Meier curve showing 60 MM patients (see Table 2) identified as having an additional copy of chromosome 1q (amp1q) using classical FISH analysis. Y-axis shows cumulative overall survival; x-axis indicates time in months. Upper line: amp1q cases treated with PAD; lower line: amp1q cases treated with VAD. Hazard Ratio=3.59, p=0.0037.

FIG. 2: Kaplan Meier curve showing 61 MM patients (see Table 2) identified as having an additional copy of chromosome 1q (amp1q) using a method according to the disclosure with all of the genes of Table 1. Y-axis shows cumulative overall survival; x-axis indicates time in months. Upper line: amp1q cases treated with PAD; lower line: amp1q cases treated with VAD. Hazard Ratio=3.96, p=0.0013.

FIG. 3: Sensitivity and specificity of all methods according to the disclosure wherein the expression level of three genes selected from Table 1 were determined. Each circle represents the results obtained with a single method according to the disclosure, wherein a combination of three genes selected from Table 1 is tested. Sensitivity was found to be between 0.567 and 0.933 and the specificity between 0.656 and 0.920. Closed circle: result obtained with all genes of Table 1.

FIG. 4: Sensitivity and specificity of all methods according to the disclosure wherein the expression levels of four genes selected from Table 1 were determined. Each circle represents the results obtained with a single method according to the disclosure, wherein a combination of four genes selected from Table 1 is tested. Sensitivity was found to be between 0.583 and 0.950 and specificity between 0.664 and 0.952. Closed circle: result obtained with all genes of Table 1.

FIG. 5: Histogram showing hazard ratios determined using every combination of three genes from Table 1. Hazard Ratios (HR) between treatment arms in the patients predicted to be amp1q of all methods according to the disclosure, wherein the expression level of three genes selected from Table 1 were determined. The histogram indicates Hazard Ratios along the x-axis, and the count along the y-axis, which is the number of three gene selections resulting in a Hazard Ratio falling in the range spanned by a particular bin (i.e., between the beginning and the end of the bar along the x-axis). The minimal HR was found to be 1.23 and the maximal HR was found to be 10.28, demonstrating that all methods according to the disclosure indicate a benefit for the proteasome inhibitor arm.

FIG. 6: Histogram showing hazard ratios determined using every combination of four genes from Table 1. Hazard Ratios (HR) between treatment arms in the patients predicted to be amp1q of all methods according to the disclosure, wherein the expression level of four genes selected from Table 1 were determined. The histogram indicates Hazard Ratios along the x-axis, and the count along the y-axis, which is the number of four gene selections resulting in a Hazard Ratio falling in the range spanned by a particular bin (i.e., between the beginning and the end of the bar along the x-axis). The minimal HR was found to be 1.14 and the maximal HR was found to be 13.34, demonstrating that all methods according to the disclosure indicate a benefit for the proteasome inhibitor arm.

### DETAILED DESCRIPTION

In the present disclosure, it is shown that subjects with multiple myeloma (MM) with an amp1q chromosomal aberration, a subgroup of subjects with multiple myeloma, respond remarkably well to treatment with a proteasome inhibitor. Also provided is a new way of diagnosing an MM subject with an amp1q chromosomal aberration based on the subject's gene expression profile.

The term “subject with multiple myeloma” or “MM subject” refers to a subject that has been diagnosed as having multiple myeloma. Results of any single test are generally not enough to diagnose multiple myeloma. Diagnosis is based on a combination of factors, including the patient's description of symptoms, the doctor's physical examination of the patient, and the results of blood tests and optional x-rays. The diagnosis of multiple myeloma in a subject may occur through any established diagnostic procedure known in the art. Generally, multiple myeloma is diagnosed when a plasma cell tumor is established by biopsy, or when at least 10% of the cells in the bone marrow are plasma cells in combination with the finding that either blood or urine levels of M protein are over a certain level (e.g., 3 g/dL and 1 g/dL, respectively) or holes in bones due to tumor growth or weak bones (osteoporosis) are found on imaging studies.

Methods for determining whether a subject with MM has an amp1q chromosomal aberration are known in the art, and any such methods may be used in aspects of this disclosure, although preferred embodiments will be disclosed herein below. It was discovered that MM patients with an amp1q chromosomal aberration respond well to proteasome inhibitors, in particular, Bortezomib, whereas subjects without the amp1q chromosomal aberration do not experience this advantageous response.

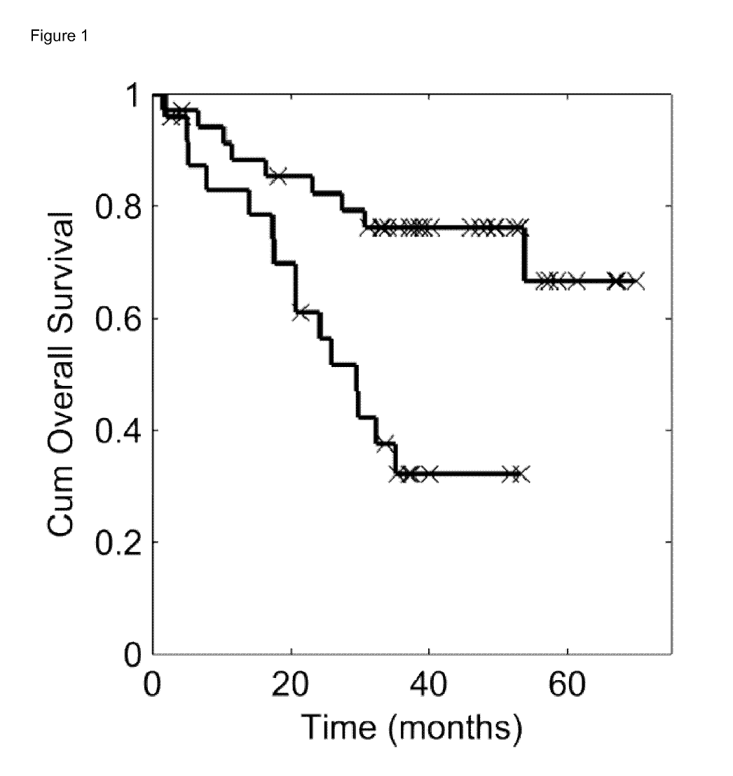

It was found that out of a total of 187 subjects with MM, 60 (32%) were classified as amp1q, using a conventional FISH analysis as described in Example 1. FIG. 1 provides a Kaplan-Meier curve wherein the top line represents amp1q cases treated with Bortezomib (a proteasome inhibitor), Adriamycin and Dexamethasone (PAD), and the bottom line represents amp1q cases treated with a conventional therapy (Vincristine Adriamycin Dexamethasone or VAD). A Hazard Ratio of 3.59 (p=0.0037) was found. It was concluded that MM patients with amp1q may be preferentially treated with a proteasome inhibitor such as Bortezomib since that provides these patients with a better life expectancy.

The disclosure, therefore, relates to a composition comprising a proteasome inhibitor for use in the treatment of a subject with multiple myeloma wherein the subject has been diagnosed with an amp1q chromosomal aberration.

In other terms, the disclosure relates to a method of treating multiple myeloma in a subject diagnosed with an amp1q chromosomal aberration wherein a composition comprising a proteasome inhibitor is administered to the subject, thereby treating multiple myeloma.

Without wishing to be bound by theory, it is put forward herein that the proteasome inhibitor for use as described herein exerts its function through its interaction with the 26S proteasome. The 26S proteasome is an essential protein complex that regulates protein degradation and protein re-localization in all cells including cancerous cells. It is involved in many cellular processes including proliferation, apoptosis, and degradation of mis-folded proteins. Furthermore, the proteasome plays a critical role in the degradation of disease-related proteins. The proteasome recognizes the ubiquitin molecule tag, which is attached to proteins by a three-step ubiquitination process.

Proteins that are targeted for degradation and re-localization are marked by an ubiquitin chain, which is recognized by the proteasome. Dependent on the localization of the ubiquitin, the protein will be processed differently by the proteasome. Proteins tagged with lysine 48-linked ubiquitin chains are marked for degradation. Proteins that are tagged with a single ubiquitin group or with lysine 63-linked chains of ubiquitin are marked for alternative biological processes including re-localization.

Degradation of protein substrates by the proteasome requires the protein to traverse the regulatory gate (19S) of the proteasome and interact with the proteolytic enzymes in the catalytic core (20S). The catalytic core particle of the proteasome forms the protein degradation machinery of the proteasome. Poly-ubiquitinated proteins (substrates) are processed in the catalytic core particle of the proteasome. The proteasome complex is currently commonly referred to as the 26S proteasome. Following gate opening, substrates translocate into the catalytic chamber of the core particle, where several active degradation sites exist.

Inhibition of the proteasome is a unique approach in cancer treatment. Preclinical activity is shown in many tumor types including solid tumors. The potential use of proteasome inhibitors in cancer treatment has been extensively described in Adams et al., Cancer Research 59:2615-2699 (1999).^{[18]} Current proteasome inhibitors bind to, and influence the catalytic core particle of the proteasome. Bortezomib or PS-341 was the first proteasome inhibitor that received FDA approval. Nowadays, other proteasome-targeted treatments are in different stages of development for application in various diseases including, but not limited to, cancer.

Although the exact downstream mechanism by which proteasome inhibitors lead to cell death of malignant cells in vitro and in vivo has not yet been fully elucidated, studies indicate that proteasome inhibitor-induced malignant cell death is associated with induction of the endoplasm reticulum, stress and activation of the unfolded protein response, inhibition of the nuclear factor kappa B inflammatory pathway, activation of caspase-8 and apoptosis, and increased generation of reactive oxygen species.

The positive effect in cancer is most likely the result of the inhibition of proteasome-regulated degradation and, therefore, accumulation of (pro-apoptotic) proteins. In addition, studies have shown that proteasome inhibitors are selective for cancer cells. Cancer cells appear to have an increased sensitivity for proteasome inhibitors; a similar effect is observed in chemotherapies.

Interfering with the 26S proteasome forms a unique approach in cancer treatment. In itself, the proteasome is a highly conserved protein complex. Furthermore, the proteasome is a relatively independent protein complex that can be described as a highly regulated trash bin mechanism for efficient protein management in all cells of the human body. As a result, downstream effects of proteasome inhibition are similar. Proteasome inhibitors inhibit the degradation machinery, followed by accumulation of proteins, which drives the elimination of tumor cells. Therefore, it is likely that a patient who would benefit from the positive effects of bortezomib treatment would also benefit from the positive effects of an alternative proteasome inhibitor.

In a preferred embodiment of the disclosure, the proteasome inhibitor is Bortezomib. Bortezomib reversibly blocks the function of the proteasome of the cell, affecting numerous biologic pathways, including those related to growth and survival of cancer cells. However, the invention also relates to a composition for a use or method as described herein wherein the proteasome inhibitor is selected from the group consisting of Bortezomib, Carfilzomib, MLN9708, Delanzomib, Oprozomib, AM-114, Marizomib, TMC-95A, Curcusone-D and PI-1840.

Bortezomib, currently has been approved for use in patients with multiple myeloma, who have already received at least one prior treatment, whose disease has worsened since their last treatment, and who have already undergone, or are unsuitable for, bone marrow transplantation. Bortezomib has significant activity in patients with relapsed multiple myeloma and MM patients that suffer from renal insufficiency.

The efficacy of Bortezomib is known to increase when used in combination with dexamethasone. Its efficacy even has shown to be improved in a synergistic way when used in combination with other drugs, such as doxorubicin.

Proteasome inhibitors may, therefore, be used in the disclosure, either alone or in combination with other drugs, selected from the group consisting of Melphalan, prednisone, doxorubicin, dexamethasone, immunomodulating drugs, monoclonal antibody drugs, including drugs based on antibody fragments, kinesin spindle protein (KSP) inhibitors, tyrosine kinase inhibitors, HDAC inhibitors, BCL2 inhibitors, Cyclin-dependent kinase inhibitors, mTOR inhibitors, heat-shock protein inhibitors, Bruton's kinase inhibitors, Insulin-like growth factor inhibitors, RAS inhibitors, PARP-inhibitors and B-RAF inhibitors.

The use of Bortezomib in combination with at least one drug selected from the group consisting of Melphalan, prednisone, doxorubicin, dexamethasone, immunomodulating drugs, monoclonal antibody drugs, including drugs based on antibody fragments, kinesin spindle protein (KSP) inhibitors, tyrosine kinase inhibitors, HDAC inhibitors, BCL2 inhibitors, Cyclin-dependent kinase inhibitors, mTOR inhibitors, heat-shock protein inhibitors, Bruton's kinase inhibitors, Insulin-like growth factor inhibitors, RAS inhibitors, PARP-inhibitors and B-RAF inhibitors is preferred.

The composition for use as described herein or the method of treatment as described herein has several advantages over prior art treatments of multiple myeloma. In the prior art treatments, Bortezomib was administered to MM patients without the pre-selection for amp1q. This resulted in the over-treatment of subjects that may not benefit from a treatment with proteasome inhibitors.

Proteasome inhibitors may cause severe peripheral neuropathy, causing pain and (severe) physical disabilities as a result, with patients even ending up in wheel chairs. Additionally, the proteasome inhibitors may be administered intravenously or subcutaneously, which can cause very high toxic doses at the site of administration. This route of administration also requires the patients to travel to a physician, which in many cases can be a serious limitation because these patients can be in poor shape and/or live far from their physicians.

The use of proteasome inhibitors is, therefore, preferably prevented in patients that would have little or no benefit from the treatment compared to other available treatments.

The disclosure, therefore, also relates to a method of treating MM in a subject, the method comprising administering to the subject a treatment regime that does not comprise a proteasome inhibitor, wherein the subject has previously been diagnosed as not having an aberrant chromosome 1q (non-amp1q).

In a preferred embodiment, the disclosure relates to a method as described above, wherein the administration of the proteasome inhibitor to the subject is made with the knowledge that the proteasome inhibitor is less effective in the treatment of MM having a non-aberrant chromosome 1q.

When applying a method according to this disclosure, patients that benefit most from the treatment (responders) may be selected and separated from patients that are less likely to benefit from the treatment (non-responders), which translates into a significant decrease of the number of patients suffering from adverse events as a result of (unnecessary) proteasome inhibitor treatment.

The method of treatment according to the disclosure thus leads to cost reduction by preventing the use of unnecessary expensive treatment, and preventing unnecessary follow-up and hospitalization of patients experiencing (serious) adverse events.

The disclosure, therefore, also relates to a method of treating MM in a subject, the method comprising administering a proteasome inhibitor to the subject, wherein the subject has previously been diagnosed as having an aberrant chromosome 1q (amp1q).

In a preferred embodiment, the disclosure relates to a method as described above, wherein the administration of the proteasome inhibitor to the subject is made with the knowledge that the proteasome inhibitor is more effective in the treatment of MM in MM subjects having an aberrant chromosome 1q (amp1q).

In other words, the disclosure relates to a method of treating MM in a subject, the method comprising:
administering to the subject a treatment regime selected from the group consisting of a treatment regime including a proteasome inhibitor and a treatment regime not including a proteasome inhibitor;
wherein the treatment regime including the proteasome inhibitor is administered to the subject where the subject has previously been determined to comprise an aberrant chromosome 1q; and
wherein the treatment regime not including the proteasome inhibitor is administered to the subject where the subject has previously been determined not to comprise an aberrant chromosome 1q.

In a preferred aspect, the disclosure relates to a method of treating a subject with MM, the method comprising subjecting a subject with MM to a treatment regime that comprises the administration of a proteasome inhibitor, wherein the subject prior to treatment has been diagnosed as having an aberrant chromosome 1q (amp1q), wherein the treatment optionally further comprises the administration of at least one drug selected from the group consisting of Melphalan, prednisone, doxorubicin, dexamethasone, immunomodulating drugs, monoclonal antibody drugs, kinesin spindle protein (KSP) inhibitors, tyrosine kinase inhibitors, HDAC inhibitors, BCL2 inhibitors, Cyclin-dependent kinase inhibitors, mTOR inhibitors, heat-shock protein inhibitors, Bruton's kinase inhibitors, Insulin-like growth factor inhibitors, RAS inhibitors, PARP-inhibitors and B-RAF inhibitors.

A new way of determining whether a subject with multiple myeloma has an amp1q chromosomal aberration was also discovered. For that, a method is provided based on gene expression analysis (Table 1). Table 1 provides a gene set for use in determining whether a subject with MM belongs to the amp1q group. The abbreviations of the genes (Gene Symbol) and the probe set are sufficient for a skilled person to unequivocally determine the relevant genes. Details may be obtained from the World Wide Web at affymetrix.com/support/technical/annotationfilesmain.affx. Details of the database are as follows: Affymetrix, netaffx-annotation-date=2012-10-15, netaffx-annotation-netaffx-build=33, genome-version=hg19, genome-version-ncbi=GRCh37.

TABLE 1
Gene set for use in a method for determining amp1q.

| | | | Negative | | Positive | |
|---|---|---|---|---|---|---|
| i | Probeset | Gene Symbol | m₀ | s₀ | m₁ | s₁ |
| 1 | 208103_s_at | ANP32E | −0.517 | 0.752 | 0.507 | 0.928 |
| 2 | 217900_at | IARS2 | −0.403 | 0.873 | 0.658 | 0.933 |
| 3 | 208684_at | COPA | −0.341 | 1.006 | 0.688 | 0.830 |
| 4 | 202374_s_at | AURKAPS1 /// | −0.465 | 1.001 | 0.583 | 0.878 |
| | | RAB3GAP2 | | | | |
| 5 | 208938_at | PRCC | −0.457 | 0.956 | 0.549 | 0.868 |
| 6 | 203073_at | COG2 | −0.344 | 0.742 | 0.596 | 1.010 |
| 7 | 210573_s_at | POLR3C | −0.209 | 0.917 | 0.791 | 0.950 |
| 8 | 212371_at | DESI2 | −0.162 | 0.910 | 0.798 | 0.881 |
| 9 | 221505_at | ANP32E | −0.426 | 0.955 | 0.532 | 0.835 |
| 10 | 219696_at | DENND1B | −0.275 | 0.832 | 0.701 | 1.005 |
| 11 | 210691_s_at | CACYBP | −0.291 | 0.959 | 0.657 | 0.834 |
| 12 | 212591_at | ARID4B /// | −0.415 | 0.982 | 0.608 | 0.975 |
| | | RBM34 | | | | |
| 13 | 201821_s_at | TIMM17A | −0.317 | 0.861 | 0.596 | 0.893 |
| 14 | 223531_x_at | GPR89A /// | −0.229 | 0.917 | 0.690 | 0.865 |
| | | GPR89B /// | | | | |
| | | GPR89C | | | | |
| 15 | 212408_at | TOR1AIP1 | −0.202 | 1.002 | 0.816 | 0.990 |
| 16 | 203033_x_at | FH | −0.345 | 0.929 | 0.617 | 0.956 |
| 17 | 211761_s_at | CACYBP | −0.399 | 0.870 | 0.558 | 1.007 |
| 18 | 225399_at | TSEN15 | −0.133 | 0.940 | 0.726 | 0.749 |
| 19 | 220642_x_at | GPR89A /// | −0.169 | 0.950 | 0.752 | 0.867 |
| | | GPR89B /// | | | | |
| | | GPR89C | | | | |
| 20 | 222140_s_at | GPR89A /// | −0.248 | 0.946 | 0.684 | 0.899 |
| | | GPR89B /// | | | | |
| | | GPR89C | | | | |
| 21 | 201275_at | FDPS | −0.071 | 0.967 | 0.835 | 0.836 |
| 22 | 212409_s_at | TOR1AIP1 | −0.205 | 0.843 | 0.669 | 0.903 |
| 23 | 209382_at | POLR3C | −0.204 | 1.009 | 0.726 | 0.866 |
| 24 | 217836_s_at | YY1AP1 | −0.469 | 1.030 | 0.508 | 0.948 |
| 25 | 214170_x_at | FH | −0.287 | 0.859 | 0.628 | 0.994 |
| 26 | 204177_s_at | KLHL20 | −0.253 | 0.892 | 0.665 | 0.984 |
| 27 | 222680_s_at | DTL | −0.459 | 0.836 | 0.401 | 0.924 |
| 28 | 211098_x_at | TMCO1 | −0.435 | 0.874 | 0.531 | 1.106 |
| 29 | 212852_s_at | TROVE2 | −0.188 | 0.996 | 0.721 | 0.880 |
| 30 | 238787_at | DENND1B | −0.342 | 0.869 | 0.481 | 0.834 |
| 31 | 235196_at | CDC73 | −0.410 | 1.015 | 0.500 | 0.879 |
| 32 | 201381_x_at | CACYBP | −0.272 | 0.897 | 0.601 | 0.920 |
| 33 | 217978_s_at | UBE2Q1 | −0.360 | 0.915 | 0.614 | 1.135 |
| 34 | 210438_x_at | TROVE2 | −0.207 | 0.955 | 0.739 | 1.036 |
| 35 | 212742_at | RNF115 | −0.243 | 0.908 | 0.647 | 0.983 |
| 36 | 218229_s_at | POGK | −0.511 | 1.058 | 0.385 | 0.846 |
| 37 | 1554351_a_at | TIPRL | −0.141 | 0.856 | 0.711 | 0.960 |
| 38 | 211609_x_at | PSMD4 | −0.225 | 0.994 | 0.645 | 0.874 |
| 39 | 200910_at | CCT3 | −0.212 | 1.005 | 0.634 | 0.812 |
| 40 | 225463_x_at | GPR89A /// | −0.190 | 0.960 | 0.673 | 0.897 |
| | | GPR89B /// | | | | |
| | | GPR89C | | | | |
| 41 | 218578_at | CDC73 | −0.449 | 0.943 | 0.412 | 0.916 |
| 42 | 203714_s_at | TBCE | −0.506 | 0.860 | 0.339 | 0.978 |
| 43 | 225400_at | TSEN15 | −0.121 | 0.940 | 0.612 | 0.671 |
| 44 | 225880_at | TOR1AIP2 | −0.381 | 0.969 | 0.553 | 1.082 |
| 45 | 221497_x_at | EGLN1 | −0.327 | 0.922 | 0.565 | 1.054 |
| 46 | 210131_x_at | SDHC | −0.118 | 0.903 | 0.739 | 0.997 |
| 47 | 218672_at | SCNM1 /// | 0.017 | 0.817 | 0.652 | 0.599 |
| | | TNFAIP8L2- | | | | |
| | | SCNM1 | | | | |
| 48 | 210460_s_at | PSMD4 | −0.142 | 0.988 | 0.667 | 0.842 |
| 49 | 216100_s_at | TOR1AIP1 | −0.253 | 1.101 | 0.548 | 0.715 |
| 50 | 219960_s_at | UCHL5 | −0.450 | 0.902 | 0.368 | 0.950 |
| 51 | 208114_s_at | ISG20L2 | −0.243 | 1.002 | 0.576 | 0.857 |
| 52 | 1554271_a_at | CENPL | −0.191 | 0.889 | 0.593 | 0.890 |
| 53 | 204788_s_at | PPOX | −0.196 | 1.018 | 0.668 | 0.953 |
| 54 | 216484_x_at | HDGF | −0.345 | 0.889 | 0.421 | 0.859 |
| 55 | 223322_at | RASSF5 | −0.503 | 0.994 | 0.360 | 0.981 |
| 56 | 203333_at | KIFAP3 | −0.343 | 0.927 | 0.521 | 1.053 |
| 57 | 200896_x_at | HDGF | −0.374 | 0.964 | 0.460 | 0.949 |
| 58 | 203715_at | TBCE | −0.400 | 1.056 | 0.372 | 0.721 |
| 59 | 203952_at | ATF6 | −0.292 | 0.991 | 0.449 | 0.732 |
| 60 | 202187_s_at | PPP2R5A | −0.361 | 0.927 | 0.456 | 0.972 |
| 61 | 203032_s_at | FH | −0.387 | 0.964 | 0.427 | 0.937 |

It was found that individuals with amp1q could be distinguished from other subjects with MM by determining the normalized expression level of at least two genes selected from the group of genes provided in Table 1, wherein the subject belongs to the amp1q group if at least two genes were overexpressed. In a preferred embodiment, the disclosure relates to a method for determining whether a subject with multiple myeloma is likely to respond to a treatment with a proteasome inhibitor or has an amp1q chromosomal aberration, comprising determining in a nucleic acid sample of a subject with multiple myeloma the expression level of a number of N genes selected from the group consisting of genes ANP32E, ARID4B///RBM34, ATF6, AURKAPS1///RAB3GAP2, CACYBP, CCT3, CDC73, CENPL, COG2, COPA, DENND1B, DESI2, DTL, EGLN1, FDPS, FH, GPR89A///GPR89B///GPR89C, HDGF, IARS2, ISG20L2, KIFAP3, KLHL20, POGK, POLR3C, PPDX, PPP2R5A, PRCC, PSMD4, RASSF5, RNF115, SCNM1///TNFAIP8L2-SCNM1, SDHC, TBCE, TIMM17A, TIPRL, TMCO1, TOR1AIP1, TOR1AIP2, TROVE2, TSEN15, UBE2Q1, UCHL5 and YY1AP1, wherein N is at least 3, and establishing that the subject is likely to respond to a treatment with a proteasome inhibitor or has an amp1q aberration in the case where at least three of the N genes are overexpressed.

Such a method may be performed using a number of techniques known in the art, such as gene sequencing, quantitative PCR, protein expression analysis and the like, but is preferably performed in a gene expression array.

In a further preferred embodiment, the disclosure is performed in a gene expression array using probes as disclosed in Table 1, i.e., selected from the group consisting of probes 208103_s_at, 217900_at, 208684_at, 202374_s_at, 208938_at, 203073_at, 210573_s_at, 212371_at, 221505_at, 219696_at, 210691_s_at, 212591_at, 201821_s_at, 223531_x_at, 212408_at, 203033_x_at, 211761_s_at, 225399_at, 220642_x_at, 222140_s_at, 201275_at, 212409_s_at, 209382_at, 217836_s_at, 214170_x_at, 204177_s_at, 222680_s_at, 211098_x_at, 212852_s_at, 238787_at, 235196_at, 201381_x_at, 217978_s_at, 210438_x_at, 212742_at, 218229_s_at, 1554351_a_at, 211609_x_at, 200910_at, 225463_x_at, 218578_at, 203714_s_at, 225400_at, 225880_at, 221497_x_at, 210131_x_at, 218672_at, 210460_s_at, 216100_s_at, 219960_s_at, 208114_s_at, 1554271_a_at, 204788_s_at, 216484_x_at, 223322_at, 203333_at, 200896_x_at, 203715_at, 203952_at, 202187_s_at and 203032_s_at.

Hence, in highly preferred embodiments of aspects of this disclosure, the presence of an amp1q chromosomal aberration, i.e., the diagnosing whether the subject has an aberrant chromosome 1q (amp1q), is established by determining the normalized expression level of at least two genes selected from the group of genes provided in Table 1, wherein the subject belongs to the amp1q group if at least two of the genes, preferably 3, 4, 5, 6, or more genes, are overexpressed.

The expression “at least two” is used herein to mean 2, 3, 4, 5, 6, 7, 8, 9, 10, 11, 12, 13, 14, 15, 16, 17, 18, 19, 20 or more, such as 25, 30, 35, 40, or more.

Determining expression levels of genes in aspects of this disclosure preferably comprises the performance of gene expression analysis on samples of a subject, preferably nucleic acid samples, such as nucleic acid samples obtained after isolating nucleic acids from tissue or fluid samples of a subject with MM. Methods for performing gene expression analysis on samples are well known in the art.

As used herein, the term “nucleic acid samples” refers to samples obtained from a subject that contain nucleic acids, such as samples obtained from blood or tissue, preferably from plasma cells.

As used herein, the term “normalized expression level” means the expression level of a gene of interest (selected from the group of genes of Table 1) divided by a reference expression level. This reference expression level or reference expression value may be arbitrarily chosen but is preferably the expression level of the gene of interest as determined in at least one control individual diagnosed with MM. Even more preferred, the reference level is the expression level of the gene of interest in a control individual diagnosed with MM that does not belong to the amp1q group. Most preferred is a reference expression level derived from a group of control individuals such as the ones described above. Such a preferred reference value may be derived by calculating the average expression level from a group of control individuals diagnosed with MM that do not belong to the amp1q group.

The expression levels of the genes according to Table 1 may be determined in DNA samples obtained from plasma cells, wherein CD138, CD319 or DC269 surface protein-positive cells are preferred.

The term “overexpressed” is used herein to indicate a level of expression that is above a reference expression level. The skilled person is familiar with methods for determining reference expression levels. In a preferred embodiment, the expression level determined in the method according to the disclosure is at least 10% above the reference value, such as 20%, 30%, 40%, 50%, 60%, 70%, 80%, 90% or even more than 100% above the reference value such as 100, 200, 300 or even 400% above the reference value.

The group of genes presented in Table 1 may, therefore, be used to determine whether a subject with MM has a chromosome 1q amplification (amp1q) or not. The expression level of any set of two genes selected from Table 1 may be determined and compared to a reference expression level for the particular gene set. If the expression level of each of the two genes is above their respective reference expression values, then the subject belongs to the chromosome 1q amplification/amp1q group.

There are a great number of suitable techniques known in the art for determining expression levels of genes. Those include, but are not limited to, gene expression array analysis, (Next generation) sequencing of RNA, RNA-FISH, quantitative-PCR, Northern Blotting, MLPA, microarray GEP, PCR, and others.

A particularly suitable method for determining the normalized expression level of the genes of Table 1 is described in Example 2. The disclosure thus relates to a method for determining whether a subject with multiple myeloma has an amp1q chromosomal aberration by determining the expression level of at least two genes selected from the group consisting of the genes listed in Table 1, wherein it is concluded that the subject has an amp1q aberration if at least two of these genes are overexpressed.

The method may even be improved by determining the expression level of more than two genes such as 3, 4, 5, 6, 7, 8, 9 or 10 genes. The method even further improves when the expression levels of 15 genes or more are determined, such as 20, 25, 30, 35, 40, or more or even all genes from Table 1.

The disclosure thus relates to a method for determining whether a subject with multiple myeloma has an amp1q chromosomal aberration by determining the expression level of three or more genes, such as all of the genes selected from the group consisting of the genes listed in Table 1, wherein it is concluded that the subject has an amp1q aberration if at least three of these genes are overexpressed.

In a further preferred embodiment, the disclosure relates to a method for determining whether a subject with multiple myeloma has an amp1q chromosomal aberration by determining the expression level of between three and all of the genes selected from the group consisting of the genes listed in Table 1, wherein it is concluded that the subject has an amp1q aberration if between three and all of the genes are overexpressed.

In machine learning and statistics, classification is the problem of identifying to which of a set of categories a new observation belongs, on the basis of a training set of data-containing observations (or instances) whose category membership is known. An algorithm that implements classification, especially in a concrete implementation, is known as a classifier.

Many classifiers are known in the art, with linear or non-linear classifier boundaries, such as, but not limited to: ClaNC, nearest mean classifier, simple Bayes classifier, linear discriminant analysis (LDA), quadratic discriminant analysis (QDA), Support Vector Machines (SVM), or the k-nearest neighbor (k-nn) classifier.

In a particularly advantageous embodiment, the disclosure relates to a method as described herein that includes a linear classifier. The ClaNC classifier (Classification to Nearest Centroids) is such a linear classifier. In that classifier, for a single MM patient called “x,” a distance d to each of the two centroids is calculated. Centroids are referred to with 0 and 1 subscripts here (e.g., reflecting non-amp1q and amp1q, respectively). The employed distance is the normalized Euclidean distance measure, resulting in a d₀ and a d₁, formulated as:

$$\begin{matrix}{d}_{0} ( x ) = \sqrt{{\sum }_{i = 1}^{N} \frac{{( {x}_{i} - {m}_{0 , i} )}^{2}}{{s}_{0 , i}^{2}}} \\ and & Formula 1 \\ {d}_{1} ( x ) = \sqrt{{\sum }_{i = 1}^{N} \frac{{( {x}_{i} - {m}_{1 , i} )}^{2}}{{s}_{1 , i}^{2}}} & Formula 2\end{matrix}$$

wherein xᵢ represents the expression level of a particular gene i of the MM patient x, N is the total number of genes or probesets used in the particular classifier, mᵢ is the mean of the centroid for gene or probeset i, and sᵢ is the standard deviation of the centroid for gene/probeset i. The MM patient is then assigned to the group with the smallest distance d (i.e., the closest centroid). An example of a determination according to a preferred embodiment of the disclosure is provided in Example 3.

The teaching as provided herein should not be interpreted so narrowly that the exact values as provided in Table 1 are the only way of arriving at the desired result. While providing the best mode of performing the disclosure when used as provided in Table 1, the numbers for m₀, m₁, s₀ and s₁ may be used as a guideline, in such a way that values that are 50% above or below these numbers will still yield satisfactory results. It should be noted in this respect that increasingly more accurate and reliable results may be obtained when the values for m₀, m₁, s₀ and s₁ resemble the values as provided in Table 1. In that respect, values that are only 10% different will provide better results than values that are 20%, 30% or 40% or more different from the values provided in Table 1.

In an alternative embodiment, the numbers may be rounded off to one or two decimals without departing from the spirit of the disclosure.

The method as described herein provides a result that correlates well with the classical methods for determining amp1q. The correlation between a method according to the disclosure using all genes from Table 1 and a classical FISH determination as described in Example 1 was established. The correlation between the classical FISH analysis and the new method is shown in Table 2.

TABLE 2
correlation matrix between classical FISH analysis
and a method according to the disclosure.

| | | FISH | | | |
|---|---|---|---|---|---|
| | | pos | neg | NA | total |
| Method | pos | 51 | 8 | 2 | 61 |
| of | neg | 9 | 117 | 0 | 126 |
| invention | | | | | |
| | total | 60 | 125 | 2 | 187 |

From the data presented in Table 2, it may be concluded that a method according to the disclosure wherein all of the genes of Table 1 are used correlates well with the classical methods such as FISH. A positive percent agreement (i.e., sensitivity) of 85%, a negative percent agreement (i.e., specificity) of 94%, a positive predictive value of 86%, and a negative predictive value of 93% were found.

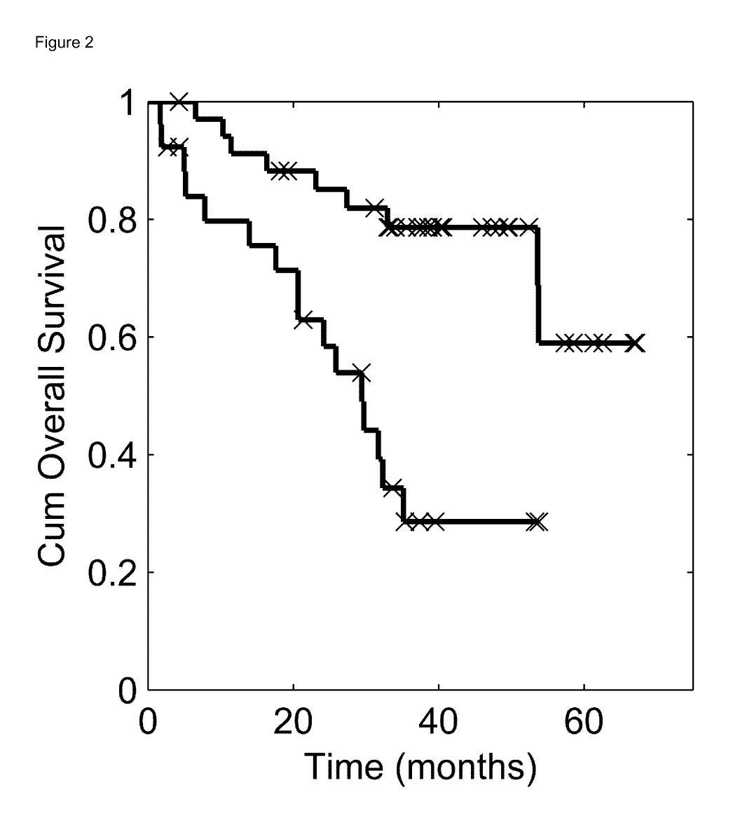

When the cumulative overall survival of the 61 patients identified as positive for amp1q in the method according to the disclosure was plotted against time in a Kaplan-Meier plot (FIG. 2), it became evident that the amp1q group as defined by the method according to the disclosure even responded better (Hazard ratio of 3.96 versus 3.59) to treatment with a proteasome inhibitor as the group defined by the classical FISH method (FIG. 1).

It may, therefore, be concluded that success was obtained in providing an objective, reproducible, easy to use, affordable diagnostic method that provides reliable and satisfactory results that even, in some preferred embodiments, outperforms the current standard, i.e., FISH analysis.

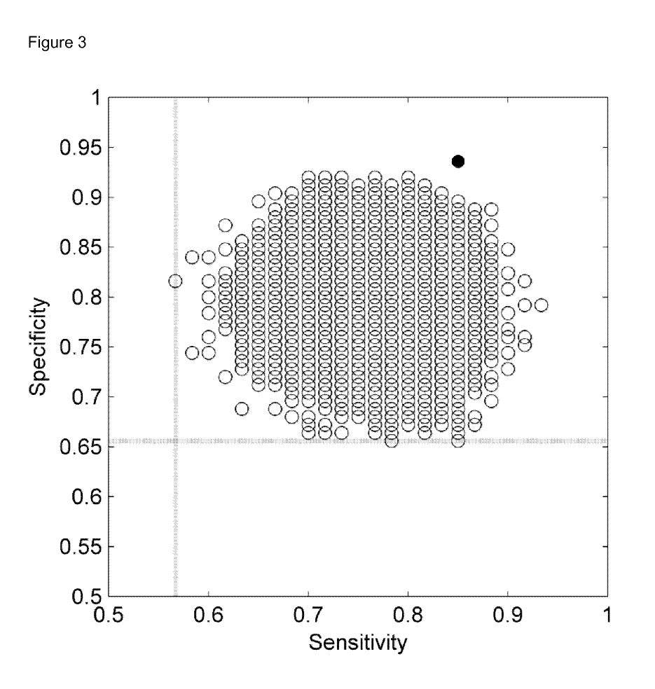

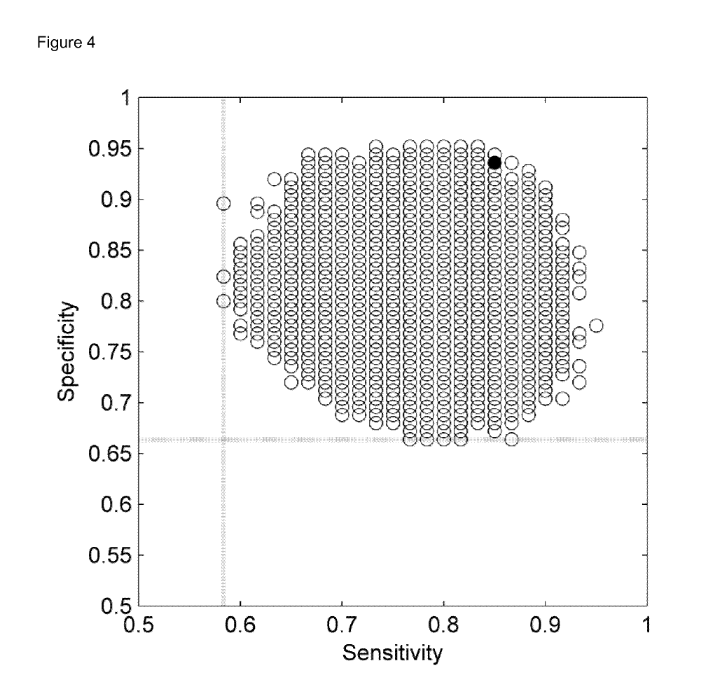

Methods according to the disclosure wherein less than all of the genes of Table 1 were tested also gave satisfactory to excellent results. FIGS. 3 and 4 show the sensitivity and specificity of such methods relative to a FISH analysis. It is evident that every combination of three or four genes from Table 1 provides better results than random selection. Random selection would have resulted in values for specificity and sensitivity of 0.5 or below, whereas both methods according to the disclosure resulted in much better sensitivities and specificities.

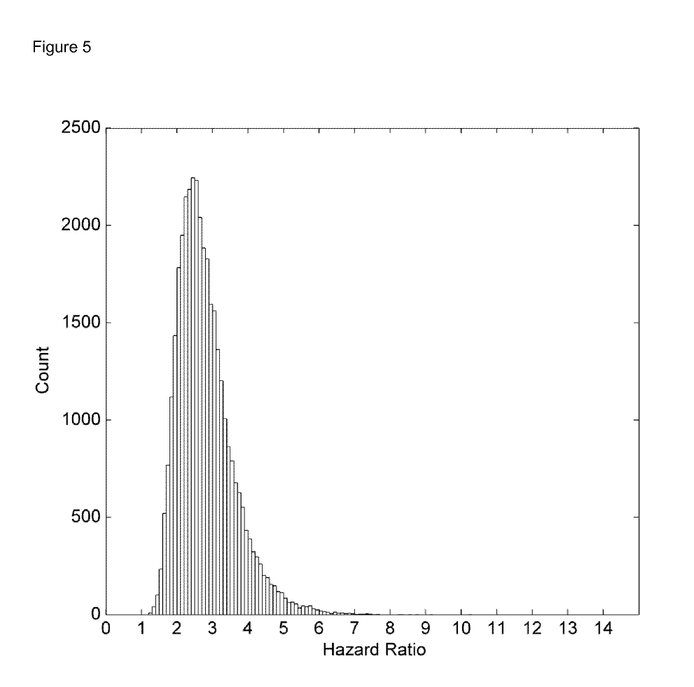

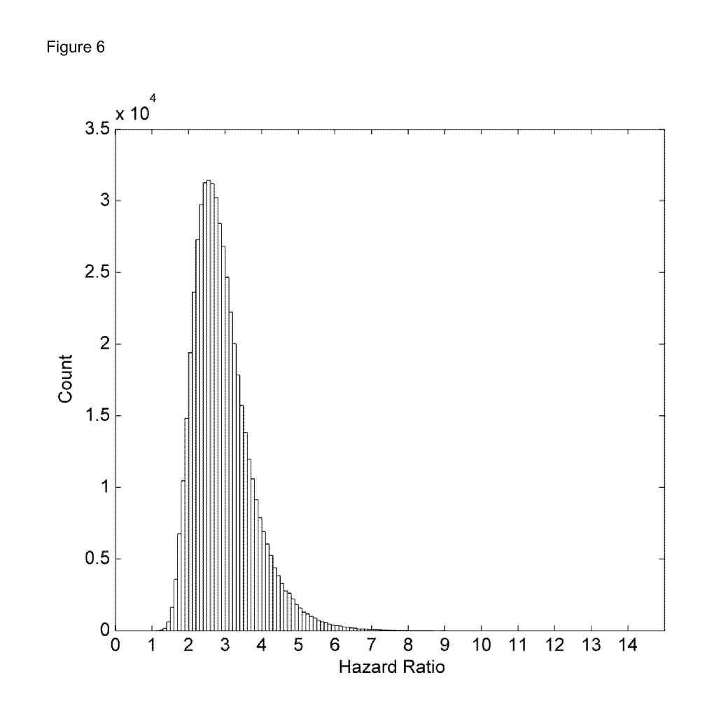

Methods according to the disclosure where less than all of the genes of Table 1 were tested for their ability to predict treatment effectiveness from proteasome inhibitors also gave satisfactory to excellent results. It was found that every combination of three or four genes from Table 1 provides a hazard ratio (HR) larger than 1, which is concordant to an improved prognosis of such subgroup when treated with a proteasome inhibitor. For a subset of three genes, the HR ranges from 1.23 to 10.28 (FIG. 5), and for a subset of four genes, the HR ranges from 1.14 to 13.34 (FIG. 6).

### EXAMPLES

### Example 1: Conventional FISH Analysis

FISH analysis was performed in 304 patients. In nonpurified plasma cell samples (n=125), at least 200 interphase nuclei per sample were analyzed by the use of epi-fluorescence microscopy and image analysis software. Within several cases, a preceding analysis of selected myeloma cells was determined by immunoglobulin light chain counterstaining or morphology. In CD138-purified PC samples (n=179), 100 nuclei were evaluated by the use of an epifluorescence microscope (Leica Microsystems). Hybridization efficiency was validated on plasma cells obtained from bone marrow of a healthy donor; thresholds for gains, deletions, and translocations were set at 10%. Detection of 1q numerical changes was performed by the use of commercial two-color probes for chromosome loci 1q21/8p21 (Poseidon Probes; Kreatech).

### Example 2: Determination of Expression Levels of Classifier Genes

In this example, the gene expression levels are determined by means of microarray technology. That is, a Bone Marrow (BM) aspirate from an MM patient is obtained, from which plasma cells are purified using immunomagnetic beads (CD138 positive; plasma cell purity of ≧80%). Subsequently, the RNA is extracted from those plasma cells, labelled cRNA constructed, and then hybridized on the Affymetrix GeneChip Human Genome U133 Plus 2.0 Array (Affymetrix, Santa Clara, Calif., USA). This chip is scanned on an Affymetrix DX2 system, providing a CEL file with measured probe intensities. This CEL file is subjected to MASS preprocessing and normalization relative to a reference cohort, which then provides the expression levels of the genes listed in Table 1.

### Example 3: Method for Determining Whether a Subject Belongs to the amp1q Cluster

In this example, the expression levels of two genes from Table 1 are determined and used to establish whether a subject belongs to the amp1q group. After determining the expression levels of the two genes, the similarity with the non-amp1q and amp1q reference groups is determined using the parameters provided in Table 1. The MM patient is then classified into the most similar group.

MM patient x appeared to have levels for ANP32E (208103_at) of 2.421 and IARS2 (217900_at) of 2.734. Using formula 1 and formula 2, d₀(x) and d₁(x) were calculated as follows.

$${d}_{0} ( x ) = \sqrt{{\sum }_{i = 1}^{N} \frac{{( {x}_{i} - {m}_{0 , i} )}^{2}}{{s}_{0 , i}^{2}}} = \sqrt{\frac{{( {x}_{1} - {m}_{0 , 1} )}^{2}}{{s}_{0 , 1}^{2}} + \frac{{( {x}_{2} - {m}_{0 , 2} )}^{2}}{{s}_{0 , 2}^{2}}} = \sqrt{\frac{{( 2.421 -- 0.517 )}^{2}}{{0.752}^{2}} + \frac{{( 2.734 -- 0.403 )}^{2}}{{0.873}^{2}}} = \sqrt{15.2640 + 12.9122} = 5.3081$$

$${d}_{1} ( x ) = \sqrt{{\sum }_{i = 1}^{N} \frac{{( {x}_{i} - {m}_{1 , i} )}^{2}}{{s}_{1 , i}^{2}}} = \sqrt{\frac{{( {x}_{1} - {m}_{1 , 1} )}^{2}}{{s}_{1 , 1}^{2}} + \frac{{( {x}_{2} - {m}_{1 , 2} )}^{2}}{{s}_{1 , 2}^{2}}} = \sqrt{\frac{{( 2.421 - 0.507 )}^{2}}{{0.928}^{2}} + \frac{{( 2.734 - 0.658 )}^{2}}{{0.933}^{2}}} = \sqrt{3.7989 + 4.9510} = 2.9580$$

Next, because d₁(x) is less than d₀(x), the sample x is called positive for amp1q. MM patient x, therefore, belongs in the amp1q cluster. This example shows the calculation for N=2, but it is trivial to extend the summation across more genes, up to all of the genes of Table 1.

### Example 4: Method for Using Subsets of Three, Four, or More Genes

In this example, the expression levels of three randomly selected genes from Table 1 are determined and used to establish whether a subject belongs to the amp1q group. After determining the expression levels of the three genes, the similarity with the non-amp1q and amp1q reference groups is determined using the parameters provided in Table 1, in analogy to the Example 3, but with N=3. The MM patient is then classified into the most similar group. Subsequently, the sensitivity and specificity are computed between the FISH label and the classification. This has been done for every subset of three genes out of all of the genes from Table 1, providing sensitivity and specificity values for every subset of three genes, as displayed in FIG. 3. At the same time, using every subset of three genes from Table 1, within the patients classified as amp1q, the Hazard Ratio between the treatment arms PAD and VAD was calculated, as shown in FIG. 5.

Analogous to the N=3 above, the same procedure was performed to test all subsets of N=4 genes from Table 1, resulting in FIGS. 4 and 6.

The same analysis may be equally applied to more than four genes, which will improve the accuracy and, hence, lead to higher sensitivities/specificities.

### REFERENCES

1. Kuehl W. M. et al. (2002) Nat. Rev. Cancer 2:175-187.
2. Fonseca R. et al. (2004) Cancer Res. 64:1546-1558.
3. Bergsagel P. L. and W. M. Kuehl (2001) Oncogene 20:5611-5622.
4. Berry N. K. et al. (2014) J. Clin. Pathol. 67(1):66-9.
5. Chng W. J. et al. (2005) Blood 106:2156-2161.
6. Kuiper R. et al. (2012) Leukemia 26:2406-13.
7. Shaughnessy J. D. et al. (2005) J. Hematology 10(Suppl 1):117-26.
8. Shaffer L. G. and N. Tommerup editors. ISCN (2005). Basel, Switzerland: Karger 2005.
9. Nilsson T. et al. (2003) Br. J. Haematol. 120:960-969.
10. Liebisch P. et al. (2003) Br. J Haematol. 122(2):193-201.
11. Morgan G. J. et al. (2012) Nat. Rev. Cancer 12(5):335-48.
12. Hanamura I. et al. (2006) Blood 108:1724-32.
13. Avet-Loiseau H. et al. (2012) Clin. Oncol. 30(16):1949-52.
14. Wu K. L. et al. (2007) Br. J. Haematol. 136(4):615-23.
15. Chng W. J. et al. (2014) Leukemia 28(2):269-77.
16. Fonseca R. et al. (2006) Leukemia 20:2034-40.
17. Alpar D. et al. (2013) Genes Chromosomes Cancer 52(9):785-93.
18. Adams et al. (1999) Cancer Research 59:2615-2699.

1. A method for determining whether a subject with multiple myeloma is likely to respond to a treatment with a proteasome inhibitor or has an amp1q chromosomal aberration, the method comprising:
determining in a nucleic acid sample of a subject with multiple myeloma the expression level of a number of N genes selected from the group consisting of genes ANP32E, ARID4B///RBM34, ATF6, AURKAPS1///RAB3GAP2, CACYBP, CCT3, CDC73, CENPL, COG2, COPA, DENND1B, DESI2, DTL, EGLN1, FDPS, FH, GPR89A///GPR89B///GPR89C, HDGF, IARS2, ISG20L2, KIFAP3, KLHL20, POGK, POLR3C, PPDX, PPP2R5A, PRCC, PSMD4, RASSF5, RNF115, SCNM1///TNFAIP8L2-SCNM1, SDHC, TBCE, TIMM17A, TIPRL, TMCO1, TOR1AIP1, TOR1AIP2, TROVE2, TSEN15, UBE2Q1, UCHL5 and YY1AP1, wherein N is at least 3, and establishing that the subject is likely to respond to a treatment with a proteasome inhibitor or has an amp1q aberration in case that at least 3 of said N genes are overexpressed.

2. The method according to claim 1, wherein the expression level of said genes is tested in a gene expression array.

3. The method according to claim 1, wherein the expression level of said genes is determined by using probes selected from the group consisting of probes 208103_s_at, 217900_at, 208684_at, 202374_s_at, 208938_at, 203073_at, 210573_s_at, 212371_at, 221505_at, 219696_at, 210691_s_at, 212591_at, 201821_s_at, 223531_x_at, 212408_at, 203033_x_at, 211761_s_at, 225399_at, 220642_x_at, 222140_s_at, 201275_at, 212409_s_at, 209382_at, 217836_s_at, 214170_x_at, 204177_s_at, 222680_s_at, 211098_x_at, 212852_s_at, 238787_at, 235196_at, 201381_x_at, 217978_s_at, 210438_x_at, 212742_at, 218229_s_at, 1554351_a_at, 211609_x_at, 200910_at, 225463_x_at, 218578_at, 203714_s_at, 225400_at, 225880_at, 221497_x_at, 210131_x_at, 218672_at, 210460_s_at, 216100_s_at, 219960_s_at, 208114_s_at, 1554271_a_at, 204788_s_at, 216484_x_at, 223322_at, 203333_at, 200896_x_at, 203715_at, 203952_at, 202187_s_at and 203032_s_at.

4. The method according to claim 1, wherein the proteasome inhibitor is selected from the group consisting of Bortezomib, Carfilzomib, MLN9708, Delanzomib, Oprozomib, AM-114, Marizomib TMC-95A, Curcusone-D and PI-1840.

5. The method according to claim 4, wherein the proteasome inhibitor is Bortezomib.

6. The method according to claim 1, wherein a linear classifier is used to determine whether a subject has an amp1q aberration.

7. The method according to claim 6, wherein the linear classifier is a ClaNC (Classification to Nearest Centroids) classifier.

8. The method according to claim 7, wherein, for a single subject x with multiple myeloma, a distance d₀ and d₁ to each of the two centroids is calculated, defined by the formulas 1 and 2:
$$\begin{matrix}{d}_{0} ( x ) = \sqrt{{\sum }_{i = 1}^{N} \frac{{( {x}_{i} - {m}_{0 , i} )}^{2}}{{s}_{0 , i}^{2}}} \\ and & Formula 1 \\ {d}_{1} ( x ) = \sqrt{{\sum }_{i = 1}^{N} \frac{{( {x}_{i} - {m}_{1 , i} )}^{2}}{{s}_{1 , i}^{2}}} & Formula 2\end{matrix}$$
wherein xᵢ represents the expression level of a particular gene i of the MM patient x according to Table 1, wherein N is the total number of genes used in the particular classifier, m₀ and s₀ are values according to Table 1, wherein mᵢ is the mean of the centroid for gene i according to Table 1 and, wherein, sᵢ is the standard deviation of the centroid for gene i according to Table 1 and, wherein, the subject with MM is then assigned to the amp1q group if the value for d₁ is less than the value for d₀ or assigned to the non-amp1q group if the value for d₀ is less than or equal to the value for d₁.

9.-12. (canceled)

13. A method for treating an individual with multiple myeloma, the method comprising:
administering a composition comprising a proteasome inhibitor to an individual determined, according to the method of claim 1, as likely to respond to a treatment with a proteasome inhibitor or determined to have an amp1q chromosomal aberration.

14. The method according to claim 13, wherein the proteasome inhibitor is selected from the group consisting of Bortezomib, Carfilzomib, MLN9708, Delanzomib, Oprozomib, AM-114, Marizomib TMC-95A, Curcusone-D, and PI-1840.

15. The method according to claim 14, wherein the proteasome inhibitor is Bortezomib.

16. The method according to claim 13, further comprising:
administering to the individual a drug selected from the group consisting of Melphalan, prednisone, doxorubicin, dexamethasone, an immunomodulating drug, a monoclonal antibody-type drug, a kinesin spindle protein (KSP) inhibitor, a tyrosine kinase inhibitor, an HDAC inhibitor, a BCL2-inhibitor, a Cyclin-dependent kinase inhibitor, an mTOR inhibitor, a heat-shock protein inhibitor, a Bruton's kinase inhibitor, an Insulin-like growth factor inhibitor, an RAS inhibitor, a PARP inhibitor, a B-RAF inhibitor, and any combination thereof.
